(19) United States
(12) Patent Application Publication
(10) Pub. No.: US 2025/0387448 A1
(43) Pub. Date: Dec. 25, 2025

(54) COMPOSITION COMPRISING HYDROLYZED ALPHA-S1-CASEIN AND A FISH PROTEIN HYDROLYSATE

(71) Applicant: VETOQUINOL SA, LURE (FR)

(72) Inventors: Michael DONABEDIAN, MAREIL-MARLY (FR); Claude BEATA, GENTILLY (FR)

(21) Appl. No.: 18/879,708

(22) PCT Filed: Aug. 25, 2023
(86) PCT No.: PCT/EP2023/073422

§ 371 Date: Dec. 27, 2024

(30) Foreign Application Priority Data
Aug. 29, 2022 (FR) FR2208642

(51) Int. Cl. A61K 38/01; A23K 10/22; A23K 10/28; A23K 20/147; A23K 50/40; A61K 9/00; A61K 9/20; A61P 25/02

(52) U.S. Cl. CPC A61K 38/018; A23K 10/22; A23K 10/28; A23K 20/147; A23K 50/40; A61K 9/0053; A61K 9/2018; A61K 9/2054; A61K 9/2059; A61P 25/02

(57) ABSTRACT

The present invention relates to a new composition comprising hydrolyzed alpha-s1-casein and a fish protein hydrolysate, its manufacturing process and the use of the composition in a food supplement, in solid or liquid form, for pets, in particular cats and dogs. Compositions according to the invention are also intended to be administered orally to help animals, preferably pets, in particular dogs or cats, overcome situations generating anxiety disorders, in particular separation-related disorders, and to regain balanced behavior. The present invention finds applications, for example, in the veterinary field, more particularly in the field of veterinary food supplements or snacks, treats or bites for pets, in particular for dogs or cats.

### TECHNICAL DOMAIN

The present invention relates to a new composition comprising hydrolyzed alpha-s1-casein and a fish protein hydrolysate, its manufacturing process and the use of the composition in a food supplement, in solid or liquid form, for pets, in particular cats and dogs.

Compositions according to the invention are also intended to be administered orally to help pets, in particular dogs or cats, overcome situations generating anxiety disorders, in particular separation-related disorders, and to regain balanced behavior.

The present invention finds applications, for example, in the veterinary field, more particularly in the field of veterinary food supplements or snacks, treats or bites for pets, in particular for dogs or cats.

### STATE OF THE ART

Dietary supplement compositions based on milk protein hydrolysate, known as hydrolyzed alpha-s1-casein, are well known. The efficacy of hydrolyzed alpha-s1-casein in helping to overcome anxiety disorders has been demonstrated in various clinical studies (CRSSA, 2002) [1].

Any change in the animal's environment, such as new people in their surroundings, lactation or weaning periods, travel, fireworks or any other change in the animal's habits, can lead to anxiety disorders. In most cases, these stressful situations can be overcome by administering compositions containing hydrolyzed alpha-s1-casein.

Hydrolyzed alpha-s1-casein, containing a bioactive peptide with relaxing properties, alpha-casozepine, has been discovered by the company INGREDIA®. Hydrolyzed alpha-s1-casein was first identified in human babies, where freshly ingested milk was found to soothe and calm newborns. The baby's enzymatic system naturally releases the alpha-casozepine responsible for this state during digestion. Research has made it possible to reproduce the baby's digestive mechanism, in particular by using the digestive enzyme “trypsin” to isolate, from casein, a milk protein hydrolysate containing the bioactive peptide alpha-casozepine.

Thus, a variety of commercial products are available today, for both humans and animals, comprising hydrolyzed alpha-s1-casein. Prior art compositions are generally solid oral forms. They can be found, for example, in capsule, tablet or powder form.

There are also compositions based on fish, mollusk and/or crustacean protein hydrolysates used in pharmaceutical and/or nutritional applications.

Separation anxiety is one of the behavioral manifestations frequently encountered by pets. It's a disorder that refers to any undesirable behavior, as well as great emotional distress, that a pet (preferably a dog or cat) exhibits when separated from its owner, or more broadly from members of its household. Undesirable behaviors include destruction, vocalization and/or inappropriate elimination. More specifically, these disorders are observed in dogs and cats (Radosta et al, 2017) [5].

In addition, pharmaceutical and/or nutritional applications based on hydrolyzed alpha-s1-casein or hydrolyzed fish, mollusk and/or crustacean proteins may prove insufficient to prevent or limit separation anxiety.

There are various, heavier, treatments for this type of disorder, but most of them are based on drugs such as antidepressants like fluoxetine or clomipramine, substances which can have side effects (Landsberg et al, 2008, 2013, Sherman et al 2008) [2-4].

There is therefore a real need today to provide effective alternative compositions to these antidepressant-based compositions.

It is to the applicant's credit that she has developed a palatable composition intended for oral administration to animals, preferably cats or dogs, comprising hydrolyzed alpha-s1-casein and a specific fish protein hydrolysate, making it possible to overcome the disadvantages of the prior art, without needing to resort to antidepressant-based treatment.

### DESCRIPTION OF THE INVENTION

The present invention thus relates to a new composition comprising:
an alpha-s1-casein hydrolysate,
a fish protein hydrolysate, the peptide distribution of the fish protein hydrolysate being:
   at least 2% by weight, relative to the total weight of peptides in the hydrolysate, of peptides with a molecular weight strictly greater than 10 kDa,
   from 25 to 80% by weight, relative to the total weight of peptides in the hydrolysate, of peptides with a molecular weight strictly greater than 1.5 kDa and less than or equal to 10 kDa,
   from 15 to 70% by weight, relative to the total weight of peptides in the hydrolysate, of peptides with a molecular weight strictly less than 1.5 kDa.

In particular, the composition according to the invention can take the form of a solid or liquid oral composition, preferably solid, powdery or non-powdery. The composition according to the invention can thus be presented in solid or liquid form, preferably in the form of a treat, a snack, a bite, a powder, a tablet or a capsule containing powder or granules.

It is to the applicant's credit that she has developed a composition comprising an alpha-s1-casein hydrolysate and a fish protein hydrolysate as described above, making it possible to prevent, regulate and/or treat, in animals, preferably pets (such as dogs, cat and/or other pets) of (anxiety) disorders such as discomfort and emotional distress, particularly in response to stressful factors and/or at least one behavioral disorder, such as one or more undesirable behaviors related to separation from its owner.

Surprisingly, this new combination according to the invention significantly increases the duration of action (thanks to the combination of metabotropic and ionotropic action) as well as the responder rate. In particular, the combined action on GABA A and B receptors has a synergistic effect on duration of action.

Advantageously, the composition according to the invention is stable, in particular hydrolyzed alpha-s1-casein and fish protein hydrolysate, which are stable for a period of at least 6 months, preferably at least 9 months or at least 12 months, and even more preferably at least 18 months.

Thus a first object of the invention is a new composition comprising:
an alpha-s1-casein hydrolysate,
a fish protein hydrolysate, the peptide distribution of the fish protein hydrolysate being:
   at least 2% by weight, relative to the total weight of peptides in the hydrolysate, of peptides with a molecular weight strictly greater than 10 kDa,
   from 25 to 80% by weight, relative to the total weight of peptides in the hydrolysate, of peptides with a molecular weight strictly greater than 1.5 kDa and less than or equal to 10 kDa,
   from 15 to 70% by weight, relative to the total weight of peptides in the hydrolysate, of peptides with a molecular weight strictly less than 1.5 kDa

### Definitions

By “alpha-s1-casein”, it is meant herein, a milk protein made up of 199 amino acid residues. Its tryptic hydrolysis releases alpha-casozepine, a bioactive peptide with relaxing, anxiolytic properties, acting like benzodiazepines. Hydrolyzed alpha-s1-casein is also known as tryptic alpha-s1-casein hydrolysate (or alpha-s1-casein hydrolysate).

Hydrolyzed alpha-s1-casein contains in particular alpha-casozepine of the following formula 1:

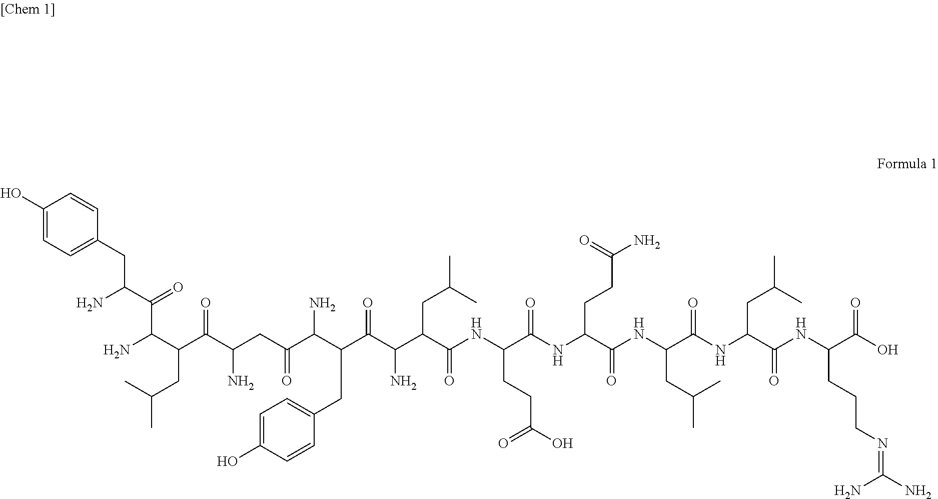 [SMILES: CC(C)CC(NC(=O)C(CC(C)C)NC(=O)C(CCC(N)=O)NC(=O)C(CCC(=O)O)NC(=O)C(CC(C)C)C(N)C(=O)C(Cc1ccc(O)cc1)C(N)C(=O)CC(N)C(=O)C(CC(C)C)C(N)C(=O)C(N)Cc1ccc(O)cc1)C(=O)NC(CCCN=C(N)N)C(=O)O]

Advantageously, hydrolyzed alpha-s1-casein is derived from milk. In particular, it is a tryptic hydrolysate of milk protein or tryptic hydrolysate of alpha-s1-casein. It is titrated in α-casozepine, a marker of the product's activity, but not 100% responsible for it (the other peptides present in the hydrolysate are also active).

Advantageously, in the composition according to the invention, the concentration of alpha-s1-casein hydrolysate ranges from 1% to 70% by weight, preferably from 1% to 60%, more preferably from 10% to 50% or even from 15% to 50%, based on the total weight of the composition.

Advantageously, the alpha-s1-casein hydrolysate can be a milk protein hydrolysate.

By “fish protein hydrolysate”, it is meant herein, a purified marine peptide obtained by enzymatic hydrolysis and whose peptide distribution is:
at least 2% by weight, relative to the total weight of peptides in the hydrolysate, of peptides with a molecular weight strictly greater than 10 kDa,
from 25 to 80% by weight, relative to the total weight of peptides in the hydrolysate, of peptides with a molecular weight strictly greater than 1.5 kDa and less than or equal to 10 kDa,
from 15 to 70% by weight, relative to the total weight of peptides in the hydrolysate, of peptides with a molecular weight strictly less than 1.5 kDa.

Advantageously, the fish protein hydrolysate can be obtained from fish containing no more than 1% lipid. The fish are preferably from the Gadidae family (Gadidae) or the Pleuronectidae family (Pleuronectidae). They may therefore be lean fish (also known as white fish) from the North Atlantic seas, preferably chosen from the Gadidae and/or Pleuronectidae families. Preferably, the fish protein hydrolysate can be obtained from cod (Gadus morhua), saithe (Pollachius virens), haddock (Melanogrammus aeglefinus) and/or plaice (Pleuronectes platessa).

Advantageously, the fish protein hydrolysate may comprise at least 80% protein, relative to the total mass of the hydrolysate. The fish protein hydrolysate may comprise lipids and minerals (ash). For example, the fish protein hydrolysate may have a respective protein/moisture/ash mass percentage distribution of >80/<5/<15. Lipids generally represent less than 0.5% of the hydrolysate mass.

Advantageously, in the composition according to the invention, the concentration of fish protein hydrolysate in the composition ranges from 5% to 20% by weight relative to the total weight of the composition, preferably from 10% to 15%.

A mass ratio of alpha-s1-casein hydrolysate to fish protein hydrolysate is defined. The said mass ratio can be in the range from 3/1 to 2/1, and is preferably equal to 2.5/1.

Advantageously, the composition according to the invention may also comprise one or more excipients (or additives). Preferably, the excipients (or additives) are chosen from texturizing agents, fillers (or diluents), oils, chelating agents, dyes, preservatives, antioxidants, flavors, and mixtures thereof.

By “texturizing agent”, it is meant herein, an additive or ingredient that improves the presentation and hold of a composition to make it more attractive. A texturizing agent gives the composition consistency while ensuring overall stability. It can be hydrophilic or hydrophobic. Hydrophobicity is the property of chemical groups that repel water. These are apolar groups that do not form hydrogen bridges with water. Conversely, hydrophilicity characterizes chemical groups that have an affinity for water. These are ionized (polar) groups that can form hydrogen bonds with water. The hydrophilic or hydrophobic properties of molecules play an important role in self-assembly phenomena in liquid media. Amphiphilic molecules possess both a hydrophobic and a hydrophilic group. Hydrophilic compounds are soluble in water, but not in fats. Conversely, hydrophobic compounds are soluble in fats and insoluble in water. They are sometimes referred to as lipophilic.

Advantageously, in the composition according to the invention, the concentration of texturizing agent is between 1% and 10% by weight, preferably 1% to 5% by weight, relative to the total weight of the composition.

Advantageously, the texturizing agent can be selected from the group comprising glycerol derivatives, e.g. glycerol, polyols, e.g. xylitol, sorbitol, polyethylene glycol, propylene glycol or polypropylene glycol, alcohols, e.g. cetyl alcohol, glycerol fatty acid esters, e.g. glycerol monostearate and acetylated glycerides, diacetyl mono glyceride, acetic esters of monoglycerides and diglycerides, lipid surfactants, e.g. sorbitan esters, glycerol monooleate, or lipids, e.g. linolein, magnesium stearate and mixtures thereof. Preferably, the texturizing agent is magnesium stearate.

By “filler” or “diluent”, it is meant herein, an additive or ingredient that increases the volume of a composition. A filler is used to fill the food preparation, i.e. to give it volume.

Advantageously, in the composition according to the invention, the concentration of filler (or diluent) is between and 80% by weight, preferably 25% to 75% by weight, relative to the total weight of the composition.

Advantageously, the filler (or diluent) can be selected from the group comprising lactose, maltodextrin, starch derivatives, e.g. pregelatinized starch or flours (rye flour, oat flour), cellulose derivatives, e.g. cellulose fiber or microcrystalline cellulose, sorbitol, mannitol, sodium lactate, waxes and maltitol and mixtures thereof. Preferably, the at least one filler or diluent is chosen from maltodextrin, lactose, mannitol, pregelatinized starch, rye flour, cellulose fiber, microcrystalline cellulose and mixtures thereof, and more preferably from maltodextrin, lactose, mannitol, microcrystalline cellulose and mixtures thereof.

By “vegetable oil”, it is meant herein, a fatty substance extracted from an oleaginous plant, i.e. a plant whose seeds, nuts or fruits contain lipids.

By “synthetic oil”, it is meant herein, an oil obtained by chemical synthesis (e.g. by polymerization, esterification or alkylation process) of components such as olefins, aromatic compounds, alcohols or acids

By “mineral oil”, it is meant herein, an oil extracted from crude oil by refining (e.g. paraffin for food use).

Advantageously, in the composition according to the invention, the oil concentration is between 1% and 20% by weight, preferably 3% to 7% by weight, relative to the total weight of the composition.

Advantageously, the oil can be chosen from a vegetable, synthetic or mineral oil.

Advantageously, the oil can be selected from the group comprising castor oil, soybean oil, rapeseed oil, peanut oil, coconut oil, avocado oil, borage oil, camelina oil, safflower oil, hemp seed oil, wheat germ oil, jojoba oil, linseed oil, macadamia oil, neem oil, black cumin oil, hazelnut oil, walnut oil, olive oil, evening primrose oil, pumpkin seed oil, grapeseed oil, perilla oil, sesame oil and sunflower oil and mixtures thereof. Preferably, the oil is chosen from the group consisting of soybean, rapeseed, sunflower or olive oil.

By “synthetic, semi-synthetic or natural plant-based flavor”, it is meant herein, any product or substance intended to be added to food products to give them an odor, taste, or odor and taste ((CE) no 1334/2008 on flavors and certain food ingredients with flavoring properties). Flavors have no nutritional qualities, but nevertheless play an essential role. Synthetic flavors are produced synthetically from chemicals. Their chemical structure and physico-chemical properties are identical to those of flavors found in nature. Flavors that do not exist in nature may be a chemical modification of a natural flavor (addition of a chemical group, replacement of one atom by another: which improves properties). Synthetic flavors fall into 3 categories (artificial flavors, processing flavors and smoke flavors). A natural plant flavor is obtained from plant raw materials, but also by physico-chemical means (from essential oils, for example). A natural flavor derived from an aromatic source must contain at least 90% of this aromatic source and 10% of natural products. The finished product is therefore an all-natural flavor. This term refers to the fact that it is a biological substance which has, however, undergone a limited number of transformations (distillation, torrefaction, cold extraction, fermentation, enzymatic reactions, enfleurage).

Advantageously, in the composition according to the invention, the concentration of synthetic, semi-synthetic or natural plant-based flavor is between 5% and 40% by weight, preferably from 10% to 30% by weight, relative to the total weight of the composition.

Advantageously, the synthetic, semi-synthetic or natural plant-based flavor, not comprising amino acids or proteins of animal origin, can be chosen from the group comprising yeast, synthetic flavors such as lard, beef, chicken, meat, veal or turkey liver, chicken liver, beef liver flavor, alone or in combination.

By “antioxidant”, it is meant herein, a substance that reduces or prevents the oxidation of other chemical substances present in the composition.

Advantageously, in the composition according to the invention, the antioxidant concentration is between 0.1% and 5% by weight, preferably from 0.2% to 1% by weight, relative to the total weight of the composition.

Advantageously, the antioxidant can be selected from the group comprising vitamin C, sodium ascorbate, calcium ascorbate, potassium ascorbate, ascorbyl palmitate, ascorbyl stearate, vitamin E and its derivatives such as alpha tocopherol, D-alpha-tocopherol, DL-alpha-tocopherol, gamma tocopherol and delta tocopherol, sodium lactate, potassium lactate, calcium lactate, disodium ethylenediaminetetraacetate, oxystearin, thiodipropionic acid, propyl gallate, octyl gallate, dodecyl gallate, ethyl gallate, guaiac resin, erythorbic acid, sodium erythorbate, potassium erythorbate, calcium erythorbate, butylhydroxyanisole, butylhydroxytoluene, ethoxyquin, sodium orthophosphate, monosodium orthophosphate, disodium orthophosphate, trisodium orthophosphate, potassium orthophosphate, monopotassium orthophosphate, dipotassium orthophosphate, tripotassium orthophosphate, calcium-dinatrium-EDTA, tin chloride, hexylresorcinol, anoxomer, dilauryl thiodipropionate, distearyl thiodipropionate, sodium thiosulfate, sodium or potassium disulfite, glucose oxidase, and mixtures thereof. Preferably, the antioxidant is selected from the group consisting of tocopherol and its derivatives (alpha tocopherol, D-alpha-tocopherol, DL-alpha-tocopherol, gamma tocopherol or delta tocopherol), butylhydroxytoluene, propyl gallate and sodium ascorbate.

By “preservative”, it is meant herein, a substance that extends the shelf life of food products by protecting them from alterations caused by microorganisms and/or protecting them against the growth of pathogenic microorganisms ((CE) no 1333/2008 on food additives). A distinction is made between organic preservatives and inorganic or mineral preservatives.

Advantageously, in the composition according to the invention, the concentration of preservative, preferably antimicrobial preservative, is between 0.05% and 1% by weight, preferably 0.1% to 0.5% by weight, relative to the total weight of the composition.

Advantageously, the preservative can be selected from the group comprising acetic acid, potassium diacetate, potassium acetate, sodium hydrogen diacetate, sodium acetate, sodium diacetate, calcium acetate, carbon dioxide, sorbic acid, sodium sorbate, potassium sorbate, calcium sorbate, benzoic acid, sodium benzoate, potassium benzoate, calcium benzoate, ethylparaben, sodium ethylparaben, propylparaben, methylparaben, sodium methylparaben, sulfur dioxide, sodium sulfite, potassium sulfite, calcium sulfite, calcium acid sulfite, potassium acid sulfite, nisin, pimaricin, hexamethyline tetramine, dimethyl dicarbonate, potassium nitrite, sodium nitrite, potassium nitrate, dehydroacetic acid, sodium dehydroacetate, propionic acid, sodium propionate, calcium propionate, potassium propionate, boric acid, borax, sodium propylparaben, heptyl para-hydroxybenzoate, ethyl lauric arginate, lecithin citrate, phytic acid and mixtures thereof. Preferably, the preservative is an antimicrobial preservative and is selected from the group consisting of potassium sorbate and benzoic acid derivatives.

Advantageously, the composition according to the invention may additionally comprise at least one dye.

Advantageously, in the composition according to the invention, the dye concentration is between 0.1% and 5% by weight, relative to the total weight of the composition.

Advantageously, the dye can be chosen from the group of food dyes. Preferably, the dye is a caramel dye.

Advantageously, the composition according to the invention may also comprise at least one chelating agent or any other excipient that may impact the texture and stability of the composition.

According to a variant of the invention, the composition according to the invention comprises:
from 1% to 60% by weight, preferably 10 to 50%, of an alpha-s1-casein hydrolysate,
from 1% to 30% by weight, preferably 5 to 20%, of a fish protein hydrolysate as defined above,
from 10% to 80% by weight of at least one bulking agent, preferably selected from lactose, microcrystalline cellulose, maltodextrin and mannitol,
from 1% to by weight of at least one texturizing agent, preferably magnesium stearate, the sum of the percentage quantities (%) being equal to 100%.

Advantageously, the composition according to the invention can be in solid or liquid form, preferably solid, in the form of powder, granules, tablets or capsules comprising powder or granules.

Advantageously, in solid form, the composition according to the invention can comprise water, preferably less than 10% water, or even less than 5%, relative to the total mass of the composition.

Advantageously, in liquid form, the invention can comprise water, preferably at least 50% water, or even more than 80%, relative to the total mass of the composition.

The invention also relates to a capsule comprising a composition according to the invention, preferably in powder or granulated form.

The invention also relates to a process or method for preparing the composition according to the invention.

Advantageously, the process or method of manufacturing a solid composition according to the invention comprises the steps:
a) Mixing the hydrolyzed alpha-s1-casein, the fish protein hydrolysate and any excipients (e.g. filler) in a paddle or ribbon mixer or in an inversion mixer, until a homogeneous powder is obtained,
b) Addition of any texturizing agents and mixing, at room temperature, until a homogeneous powder is obtained,
c) Extrusion of the homogeneous powder obtained in step b) to produce snacks, bites or treats,
d) Compression of the homogeneous powder obtained in step b) to obtain tablets, or
e) Capsuling the homogeneous powder obtained in step b).

Steps c), d) and e) are alternative steps, and are not implemented in the same process. The process can thus be carried out according to the ordered sequence of steps a-b-c, a-b-d or a-b-e.

Advantageously, step a) is carried out at room temperature, i.e. a temperature in the range 15 to 25° C. Preferably step b) is carried out under identical temperature conditions.

Steps b) and c) can be carried out in a mixer equipped with extrusion blades of the deflocculating propeller or marine propeller type.

The composition according to the invention is intended for oral administration.

The invention also relates to a food supplement comprising a composition according to the invention. The food supplement may be selected from veterinary food supplements, in the form of a treat, snack, bite, powder, tablet or capsule containing powder or granules, for pets, for example dogs and/or cats.

The invention also includes the use of a combination of alpha-s1-casein hydrolysate and fish protein hydrolysate in a solid or liquid, preferably solid, food supplement. Alpha-s1-casein hydrolysate and fish protein hydrolysate are as defined above. Preferably, the alpha-s1-casein hydrolysate/fish protein hydrolysate weight ratio is in the range from 3:1 to 2:1, and is preferably equal to 2.5:1.

Advantageously, in the use according to the invention, the food supplement comprises at least one excipient (or additive) selected from texturizing agents, fillers (or diluents), oils, chelating agents, dyes, preservatives, antioxidants, flavors, and mixtures thereof.

Advantageously, the food supplement, solid or liquid, is a treat, snack or bite for animals, preferably pets, even more preferably cats or dogs.

The invention also relates to a composition according to the invention intended for oral administration to animals, preferably domestic animals, in particular cats or dogs.

Thus, the invention also relates to a composition according to the invention for its use in helping an animal, preferably a pet, in particular a dog or cat, to overcome stress-generating situations and regain balanced behavior, for its use in the management and/or prevention of anxiety disorders in an animal, preferably a pet, in particular a dog or cat.

The invention further relates to a use of a composition according to the invention to prevent or alleviate anxiety disorders in animals, preferably in cats or dogs.

Further advantages may become apparent to the skilled person upon reading the examples below, illustrated by the appended figures, which are given by way of illustration and not by way of limitation

### EXAMPLES

### Example 1: Manufacturing Processes for Compositions According to the Invention

The example compositions are made from the following compounds:
Hydrolyzed alpha-s1-casein (Lactium®),
Fish protein hydrolysate,
Magnesium stearate,
Lactose,
Maltodextrin,
Microcrystalline cellulose, and
Mannitol.

Table 1 below shows the composition of the protein component of the fish protein hydrolysate:

TABLE 1
Peptide distribution of the fish protein hydrolysate

| | >10,000 Da | 1500 < >10000 | <1500 Da |
|---|---|---|---|
| Wavelength | (%) | Da | (%) |
| 280 nm | 5.8 | 27.4 | 66.7 |
| | 9.2 | 28.6 | 62.3 |
| | 6.7 | 29.3 | 63.9 |
| 214 nm | 6 | 74.1 | 19.8 |
| | 5.7 | 73.3 | 20.3 |
| | 4.2 | 75.6 | 20.1 |

This distribution was measured using the LC-SEC UV steric exclusion chromatography analysis method. A 200 A column 15*7.8 mm*1.7 μm was used. Separation was carried out in a 20 min isocratic LC method at 0.850 μl/min in a buffer consisting of 0.05 sodium phosphate and 0.075 sodium chloride at pH=7.2.

Fish protein hydrolysate is derived from a blend of cod (Gadus morhua), saithe (Pollachius virens), haddock (Melanogrammus aeglefinus) and/or plaice (Pleuronectes platessa).

The fish protein hydrolysate has a respective protein/moisture/ash mass percentage distribution of >80/<5/>15. Lipids represent less than 0.5% of the hydrolysate mass.

Protein, moisture and ash contents are measured in accordance with standards NF V 04-407, NF V 04-401 and NF V 04-404 respectively.

### Preparing the Powder Mixture:

a) The filler(s) (e.g. lactose, cellulose, maltodextrin, mannitol), hydrolyzed alpha-s1-casein and fish protein hydrolysate are successively added at room temperature to an inversion mixer and then mixed.

The resulting powder mix is homogeneous and free from agglomerates.

### Lubricating the Powder Mixture:

b) In a suitable container, the powder mixture previously obtained and the texturizing agent (i.e. magnesium stearate) are mixed, at room temperature, using an inversion mixer

Stirring is maintained until a homogeneous powder is obtained.

### Capsule Filling

The resulting homogeneous mixture is fed into a capsule filler to be filled into capsules containing 75, 225 or 450 mg of Lactium®.

Composition 1 is obtained:

TABLE 2
Composition 1

| Composition 1 | Centesimal formula (weight %) |
|---|---|
| Lactium ® | 39.2 |
| Fish protein hydrolysate | 15.68 |
| Maltodextrin | 40.12 |
| Magnesium stearate | 5 |

### Example 2: Compositions According to the Invention

Compositions 2 to 11 were prepared according to the process described in Example 1:

TABLE 3
composition 2 to 11

| | Centesimal formula (weight %) |
|---|---|
| Composition 2 | |
| Lactium ® | 37.42 |
| Fish protein hydrolysate | 14.97 |
| Microcrystalline cellulose | 42.62 |
| Magnesium stearate | 4.99 |
| Composition 3 | |
| Lactium ® | 29.68 |
| Fish protein hydrolysate | 11.87 |
| Maltodextrin | 54.5 |
| Magnesium stearate | 3.96 |
| Composition 4 | |
| Lactium ® | 29.1 |
| Fish protein hydrolysate | 11.64 |
| Mannitol | 55.39 |
| Magnesium stearate | 3.88 |
| Composition 5 | |
| Lactium ® | 12.5 |
| Fish protein hydrolysate | 6.25 |
| Lactose | 79.25 |
| Magnesium stearate | 2 |
| Composition 6 | |
| Lactium ® | 20 |
| Fish protein hydrolysate | 10 |
| Mannitol | 67.5 |
| Magnesium stearate | 2.5 |
| Composition 7 | |
| Lactium ® | 44.6 |
| Fish protein hydrolysate | 17.84 |
| Maltodextrin | 32.56 |
| Magnesium stearate | 5 |
| Composition 8 | |
| Lactium ® | 67.5 |
| Fish protein hydrolysate | 22.5 |
| Microcrystalline cellulose | 7 |
| Magnesium stearate | 3 |
| Composition 9 | |
| Lactium ® | 14.7 |
| Fish protein hydrolysate | 5.88 |
| Maltodextrin | 74.42 |
| Magnesium stearate | 5 |
| Composition 10 | |
| Lactium ® | 27.3 |
| Fish protein hydrolysate | 9.1 |
| Lactose | 62.1 |
| Magnesium stearate | 1.5 |
| Composition 11 | |
| Lactium ® | 25.61 |
| Fish protein hydrolysate | 10.25 |
| Lactose | 60.72 |
| Magnesium stearate | 3.42 |

### Example 3: Assessment of Separation Disorders in Cats

A test to evaluate owner separation-related disorders was carried out on 100 cats of different weights and breeds. Each cat was given two capsules of composition 9, each containing 75 mg Lactium®, once a day for 30 days (D1 to D30)

Experimentation and, more specifically, characterization of the disorders measured are based on Radosta [5].

The behavioral disorders assessed as a result of the animal's separation from its owners are as follows:
Constant meowing;
Excessive licking;
Scratching and destruction;
Urination in inappropriate places;
Urine marking;
Defecation.

The study was carried out in two phases:

Phase 1 (at DO): assessment of the frequency of onset of separation-related disorders in the animal for 24H by the owner according to one of the following criteria:
Not affected (N),
Occasionally (D),
Often (S),
All the time (T).

Phase 2 (at D15 and D30): measurement of improvement in the animal's condition over 24 hours, depending on whether the owner observes:
No improvement (−),
Average improvement (+),
Good improvement (++),
Excellent improvement (+++).

The results obtained in Phase 1 are presented in Table 4 below:

TABLE 4
Results of separation disorders assessment in cats (t = D 0).

| | | Scratching | Micturition in | | | |
|---|---|---|---|---|---|---|
| N = | Constant | and | inappropriate | Urine | | Excessive |
| 100 | meowing | destruction | places | marking | Defecation | licking |
| N | 24% | 49% | 58% | 69% | 79% | 47% |
| D | 43% | 23% | 18% | 12% | 15% | 19% |
| S | 24% | 15% | 17% | 13% | 2% | 23% |
| T | 9% | 13% | 7% | 6% | 4% | 11% |

The results obtained in phase 2 are presented in tables 5 and 6 below:

TABLE 5
Percentage of individuals per behavior observed at t = D 15 for 24 H.

| | | Scratching | Micturition in | | | |
|---|---|---|---|---|---|---|
| | Constant | and | inappropriate | Urine | | Excessive |
| N = | meowing | destruction | places | marking | Defecation | licking |
| 100 | (% of ind.) | (% of ind.) | (% of ind.) | (% of ind.) | (% of ind.) | (% of ind.) |
| − | 13 | 10 | 9 | 0 | 0 | 13 |
| + | 44 | 52 | 23 | 20 | 20 | 42 |
| ++ | 38 | 29 | 46 | 55 | 40 | 32 |
| +++ | 4 | 10 | 23 | 25 | 40 | 13 |

TABLE 6
Percentage of individuals per behavior observed at t = D 30 for 24 H.

| | | Scratching | Micturition in | | | |
|---|---|---|---|---|---|---|
| | Constant | and | inappropriate | Urine | | Excessive |
| N = | meowing | destruction | places | marking | Defecation | licking |
| 100 | (% of ind.) | (% of ind.) | (% of ind.) | (% of ind.) | (% of ind.) | (% of ind.) |
| − | 5 | 11 | 3 | 14 | 0 | 7 |
| + | 45 | 36 | 27 | 19 | 27 | 33 |
| ++ | 34 | 36 | 47 | 48 | 40 | 41 |
| +++ | 16 | 18 | 23 | 19 | 33 | 19 |

On average, 90% of cats experienced an improvement in their condition.

Furthermore, there was an increase in this improvement at D30 compared with D15. For example, in the case of constant meowing, 95% improvement was observed at D30, compared with 87% at D30.

Thus, composition 9 improved problems associated with separation from the owner at D15 and D30. Surprisingly, there was an increase in improvement as the treatment time increased. No negative effects on behavior were observed.

### Example 4: Evaluation of Separation Disorders in Dogs

A test to evaluate owner separation-related disorders was conducted on 300 dogs of different weights and breeds. Each dog was administered once a day
two capsules of composition 9, each containing 75 mg of Lactium® for dogs weighing between 5 and 10 kg,
one capsule of composition 1, each containing 225 mg Lactium® for dogs weighing between 10 and 15 kg,
one capsule of composition 7, each containing 450 mg Lactium® for dogs weighing between 15 and 30 kg, and two capsules for dogs weighing between 30 and 60 kg, for 30 days (from D1 to D30).

Experimentation and, more specifically, characterization of the disorders measured are based on Radosta [5].

The behavioral problems associated with separation from the animal assessed are as follows:
The animal constantly follows the owner,
Anxious behavior when owner leaves
Destruction,
Urination/defecation,
Excessive greeting,
Barking/whining.

The study was conducted in two phases:

Phase 1 (at D0): evaluation the frequency of onset of separation-related disorders in the animal with its owner for 24 H according to the following criteria:
Not affected (N),
Occasionally (D),
Often (S),
All the time (T).

Phase 2 (at D15 and D30): measurement of the improvement in the animal's symptoms over 24 hours by the owner, depending on whether he observes:
No improvement (−),
Average improvement (+),
Good improvement (++),
Excellent improvement (+++).

The results obtained in phase 1 are presented in Table 7 below:

TABLE 7
Percentage of individuals per behavior observed at t = D 0 for 24 H.

| | | Anxious | | | | |
|---|---|---|---|---|---|---|
| | | behavior | | | | |
| N = | Owner | when the | | Micturition/ | Excessive | Barking/ |
| 300 | follow-up | owner leaves | Destruction | defecation | greetings | moaning |
| N | 1% | 2.60% | 48.30% | 55.70% | 4.30% | 14.30% |
| D | 10% | 12.70% | 27.30% | 22.30% | 16.40% | 33.30% |
| S | 36% | 30.70% | 14.70% | 13% | 29% | 26.70% |
| T | 52% | 54% | 9.70% | 9% | 50.30% | 24.30% |

The results obtained in phase 2 are presented in tables 8 and 9 below:

TABLE 8
Percentage of individuals per behavior observed at t = D 15 for 24 H.

| | | Anxious | | | | |
|---|---|---|---|---|---|---|
| | | behavior | | | | |
| | Owner | when the | | Micturition/ | Excessive | Barking/ |
| N = | follow-up | owner leaves | Destruction | defecation | greetings | moaning |
| 300 | (% of ind.) | (% of ind.) | (% of ind.) | (% of ind.) | (% of ind.) | (% of ind.) |
| − | 23 | 17 | 17 | 28 | 27 | 20 |
| + | 41 | 44 | 36 | 21 | 34 | 45 |
| ++ | 31 | 34 | 31 | 32 | 32 | 28 |
| +++ | 5 | 5 | 16 | 19 | 7 | 8 |

TABLE 9
Percentage of individuals per behavior observed at t = D 30 for 24 H

| | | Anxious | | | | |
|---|---|---|---|---|---|---|
| | | behavior | | | | |
| | Owner | when the | | Micturition/ | Excessive | Barking/ |
| N = | follow-up | owner leaves | Destruction | defecation | greetings | moaning |
| 300 | (% of ind.) | (% of ind.) | (% of ind.) | (% of ind.) | (% of ind.) | (% of ind.) |
| − | 18 | 13 | 8 | 19 | 24 | 21 |
| + | 32 | 39 | 27 | 32 | 34 | 36 |
| ++ | 38 | 33 | 48 | 28 | 32 | 32 |
| +++ | 12 | 15 | 16 | 21 | 10 | 11 |

On average, 80% of dogs showed an improvement in their condition.

Furthermore, there was an increase in this improvement at D30 compared with D15. For example, for the destruction disorder, there was a 92% improvement at D30 versus 83% at D30. For urine/defecation disorders, there was an 81% improvement at D30 compared with 72% at D15.

The combination of alpha-s1-casein hydrolysate and fish protein hydrolysate showed an improvement in separation-related disorders at D15 and D30. Moreover, the improvement in these disorders increased as the duration of treatment increased. No negative effects on behavior were observed.

### REFERENCES

1. Lanoir D, Canini F, Messaoudi M, Lefranc-Millot C, Demagny B et al. Long term effects of a bovine milk alpha-S1 casein hydrolysate on healthy low and high stress responders. 2002, Stress, Vol 5 (suppl.), page 124
2. Landsberg G. M, Hunthausen W. L, and Ackerman L. J. Behavior problems of the dog and cat. 2013, Third edition. Edinburgh: Saunders/Elsevier. ISBN 978-0-7020-4335-2. SF433
3. Landsberg G. M, Melese P, Sherman B. L, Neilson J. C, Zimmerman A and Clarke T. P. Effectiveness of fluoxetine chewable tablets in the treatment of canine separation anxiety. 2008 January, Journal of Veterinary Behavior, Vol. 3, no 1, pages 12-19.
4. Sherman B. L Separation anxiety in dogs. 2008 January, Compendium on Continuing Education for the Practising Veterinarian-North American Edition, 30 (1), pages 27-42.
5. Radosta L and Fagen A. Separation-Related Disorders and the Differences Between Dogs and Cats. 2017, Today's Veterinary Practice, Vol 7, no 3, pages 53-58

1. Composition comprising:
an alpha-s1-casein hydrolysate,
a fish protein hydrolysate, the peptide distribution of the fish protein hydrolysate being:
at least 2% by weight, relative to the total weight of peptides in the hydrolysate, of peptides with a molecular weight strictly greater than 10 kDa,
from 25 to 80% by weight, relative to the total weight of peptides in the hydrolysate, of peptides with a molecular weight strictly greater 1.5 kDa and less than or equal to 10 kDa,
from 15 to 70% by weight, relative to the total weight of peptides in the hydrolysate, of peptides with a molecular weight strictly less than 1.5 kDa.

2. Composition according to claim 1, wherein the fish protein hydrolysate is obtained from cod (Gadus morhua), saithe (Pollachius virens), haddock (Melanogrammus aeglefinus) and/or plaice (Pleuronectes platessa).

3. Composition according to claim 1, wherein the alpha-s1-casein hydrolysate/fish protein hydrolysate weight ratio is in the range from 3:1 to 2:1, preferably equal to 2.5:1.

4. Composition according to claim 1, wherein the concentration of alpha-s1-casein hydrolysate ranges from 1% to 70%, preferably from 1 to 60%, more preferably from 10% to 50% or even from 15 to 50%, relative to the total mass of the composition.

5. Composition according to claim 1, wherein the alpha-s1-casein hydrolysate is an alpha-s1-casein tryptic hydrolysate.

6. Composition according to claim 1, wherein the concentration of fish protein hydrolysate is in the range from 5% to 20% by weight relative to the total weight of the composition, preferably from 10% to 15%.

7. Composition according to claim 1, further comprising an excipient, preferably selected from texturizing agents, fillers, oils, chelating agents, dyes, preservatives, antioxidants, flavors, and mixtures thereof.

8. Composition according to claim 1, wherein the filler is chosen from lactose, maltodextrin, pregelatinized starch, flours, cellulose fiber, microcrystalline cellulose, sorbitol, mannitol, sodium lactate, waxes and maltitol and mixtures thereof.

9. Composition according to claim 1, wherein the filler concentration is in the range from 10% to 80% by weight relative to the total weight of the composition.

10. Composition according to claim 1, in which the texturizing agent is chosen from glycerin, xylitol, sorbitol, polyethylene glycol, propylene glycol, polypropylene glycol, cetyl alcohol, glycerol monostearate, acetylated glycerides, diacetyl mono glyceride, acetic esters of monoglycerides and diglycerides, sorbitan esters, glycerol monooleate, linolein, magnesium stearate and mixtures thereof.

11. Composition according to claim 1, in which the concentration of texturizing agent is in the range from 1% to 10% by weight relative to the total weight of the composition.

12. A food supplement comprising a composition according to claim 1, in liquid or solid form, preferably a treat, snack, bite, powder, tablet or capsule containing powder or granules.

13. Composition or food supplement according to claim 1 for use in the management and/or prevention of anxiety disorders in animals, preferably pets, preferably dogs or cats.

14. Use of a composition or food supplement according to claim 1 to prevent or alleviate anxiety disorders in an animal, preferably pets, preferably selected from dogs and cats, related to separation from its owner.

15. Use of a combination of alpha-s1-casein hydrolysate and fish protein hydrolysate as defined in claim 1 in a food supplement for animals, preferably pets, preferably dogs or cats.
